(19) United States
(12) Patent Application Publication
(10) Pub. No.: US 2010/0173869 A1
(43) Pub. Date: Jul. 8, 2010

(54) TREATMENT OF SURFACTANTS

(76) Inventors: Matthias W. Amrein, Calgary (CA); Lasantha C. Gunasekara, Calgary (CA)

Correspondence Address: Jaekle Fleischmann & Mugel, LLP, 12 Fountain Plaza, Buffalo, NY, 14202-2292, US

(21) Appl. No.: 12/628,544

(22) Filed: Dec. 1, 2009

Related U.S. Application Data
(63) Continuation of application No. PCT/EP2008/004274, filed on May 29, 2009.

(30) Foreign Application Priority Data
Jun. 1, 2007 (DE) 10 2007025 898 A1

(51) Int. Cl. A61K 31/715; A61P 11/00

(52) U.S. Cl. 514/58

(57) ABSTRACT

The invention relates to a method for treatment of a surfactant, in particular a pulmonary surfactant. The method is further established in that the surfactant is treated with a lipid sequestrating or cholesterol sequestrating surfactant treatment agent, in which given, in particular neutral lipids or cholesterol are selectively sequestrated by means of the surfactant treatment agent, such that the effect of the lipids and/or the effect of the cholesterol on the surfactant is reduced or reversed.

The invention further relates to a method for producing a surfactant treatment agent, in particular a pulmonary surfactant treatment agent. The invention also relates to a surfactant treatment agent and a use of a treatment agent for treating a surfactant, in particular a pulmonary surfactant and a use of cyclodextrins.

### FIELD OF THE INVENTION

This application is a National Phase filing under 35 U.S.0 371 of PCT Appl. No. PCT/EP2008/004274 filed May 29, 2009, which claims the benefit of German Application No. DE 10 2007025898 A1 filed on Jun. 1, 2007, both applications of which application are incorporated by reference herein in their entirety

### BACKGROUND OF THE INVENTION

The invention relates to a method for enhancing a surfactant, in particular a pulmonary surfactant, as well as a method for producing a surfactant enhancement agent, in particular a pulmonary surfactant enhancement agent. The invention also relates to a surfactant enhancement agent and a use of an enhancement agent for enhancing a surfactant, in particular a pulmonary surfactant and a use of cyclodextrin.

It is known that the pulmonary surfactant is a surface-active substance lining the respiratory system of the lung. Type II pneumocytes produce the surfactant, store it as lamellar bodies and finally express it in the alveolar liquid phase. From there it spreads as a molecular thin film on the air/water boundary surface. It displays its effect by dynamically adapting the surface tension of the boundary surface to the current extension of the boundary surface during the breathing cycle.

The pulmonary surfactant consists of lipids—primarily lecithin (DPPC), unsaturated phosphatidylcholines and negatively charged phosphatidylglycerols—and proteins. Two of the four surfactant-specific proteins, SP-B and SP-C, are responsible for the surface-active properties of the surfactant in interaction with the lipids.

In 1959 it was shown that the lack of pulmonary surfactant in the lungs of premature infants leads to respiratory distress syndrome (RDS), which at that time was the most frequent cause of death for premature infants. For about the past 15 years, extracts from the lungs of cattle or pigs have been used as a medication to treat RDS. Animal preparations are generally expensive and carry the risk of transmitting infectious diseases.

The surfactant (short for surface active agent) consists of glycero-phospholipids, specific proteins, neutral fats and cholesterol. The surfactant covers the alveolar surface and reduces the surface tension, so that after birth the alveoli do not collapse in the human body during exhalation.

Insufficient function of the surfactant can be the cause of respiratory insufficiency, known as Infant Respiratory Distress Syndrome (IRDS) in premature infants and newborns or in adults as Adult Respiratory Distress Syndrome (ARDS). These lung illnesses are the result of a surfactant deficiency, which lead to an inadequate expansion of the lungs (atelectasis) after a collapse of the pulmonary alveoli.

The lung surfactant consists of 90% lipids and 10% proteins. Although cooperation between the surfactant-specific proteins and the lipids is necessary for a completely functional respiratory process, the lipids are essential for the vitally important reduction of surface tension.

The function of pulmonary surfactants and their inhibited function with pulmonary infections and pulmonary diseases have been described in numerous publications. In this regard reference is made to the publications:

T. R. Martin, Cytokines and the Acute Respiratory Distress Syndrome (ARDS): a question of balance, Nat. Med. 3 (1997), pp. 272.

Artigas, G. R. Bernard, J. Cadet, D. Dreyfuss, L. Gattinoni, The American-European consensus conference on ARDS, part 2: ventilatory, pharmacologic, supportive therapy, study design strategies, and issues related to recovery and remodeling. Acute respiratory distress syndrome, Am. J. Respir. Crit. Care Med. 157 (Pt 1) (1998), pp. 1332.

G. R. Bernard, A. Artigas, K. L. Brigham, J. Carlet, K. Falke, The American-European consensus conference on ARDS: definitions, mechanisms, relevant outcomes, and clinical trial coordination, Am. J. Respir. Crit. Care Med. 149 (1994), pp. 818.

A. B. Montgomery, M. A. Stager, C. J. Carrico, E. D. Hudson, Causes of mortality in patients with the adult respiratory distress syndrome, Am. Rev. Respir. Dis. 132 (1985), pp. 485.

G. Karagiorga, G. Nakos, E. Galiatsou, M. E. Lekka, Biochemical parameters of bronchoalveolar lavage fluid in fat embolism, Intensive Care Medicine 32 (2006), pp. 116-123.

L. D. Hudson, K. P. Steinberg, Epidemiology of acute lung injury and ARDS, Chest 116 (1999), pp. 74S.

G. Devendra, R. G. Spragg, Lung surfactant in subacute pulmonary disease, Respir. Res. 3 (2002), pp. 19.

M. Griese, R. Essl, R. Schmidt, E. Rietschel, F. Ratjen, M. Ballmann, K. Paul, Pulmonary surfactant, lung function, and endobronchial inflammation in cystic fibrosis, American Journal Of Respiratory And Critical Care Medicine 170 (2004), pp. 1000-1005.

M. Griese, L. Felber, K. Reiter, R. Strong, K. Reid, B. H. Belohradsky, G. Jager, T. Nicolai, Airway inflammation in children with tracheostomy, Pediatr. Pulmonol. 37 (2004), pp. 356-361

Pulmonary surfactants form a complex film which has or plays a critical role in the reduction of surface tension in the respiratory tract in the hydrated air/lung interface. With infectious lung diseases, the molecular profile of pulmonary surfactants in the alveoli and respiratory tract is changed so that the pulmonary surfactant film is quite a lot less effective or ineffective for reducing the surface tension, which is accompanied by a strong decrease in the area available for gas exchange.

Furthermore, it is known that cholesterol and/or or an elevated level of cholesterol in the surfactants are a primary cause of surfactant dysfunction. In this regard reference is also made to the following publications:

J. E. Lewis, R. Veldhuizen, The role of exogenous surfactant in the treatment of acute lung injury, Annual Review Of Physiology 65 (2003), 613-642

H. W. Taeusch, K. M. W. Keough, Inactivation of pulmonary surfactant and the treatment of acute lung injury, Pediatric Pathology and Molecular Medicine 20 (2001), 519-536

H. W. Taeusch, J. B. de la Serna, J. Perez-Gil, C. Alonso, J. A. Zasadzinski, Inactivation of pulmonary surfactant due to serum-inhibited adsorption and reversal by hydrophilic polymers: Experimental, Biophysical Journal 89 (2005), 1769-1779

K. Rodriguez-Capote, D. Manzanares, T. Haines, F. Possmayer, Reactive oxygen species inactivation of surfactant involves structural and functional alterations to surfactant proteins SP-B and SP-C, Biophysical Journal 90 (2006), 2808

S. Andersson, A. Kheiter, T. A. Merritt, Oxidative inactivation of surfactants, Lung 177 (1999), 179

L. Mark, E. P. Ingenito, Surfactant function and composition after free radical exposure generated by transition metals, Am. J. Physiol.-lung Cell. Mol. Physiol. 276 (1999), L491

N. Gilliard, G. P. Heldt, J. Loredo, H. Gasser, H. Redl, T. A. Merritt, R. G. Spragg, Exposure of the hydrophobic components of porcine lung surfactant to oxidant stress alters surface tension properties, Journal Of Clinical Investigation 93 (1994), 2608

L. Gunasekara, S. Schurch, W. M. Schoel, K. Nag, Z. Leonenko, M. Haufs, M. Amiein, Pulmonary surfactant function is abolished by an elevated proportion of cholesterol, Biochimica Et Biophysica Acta 1737 (2005), 27-35.

H. Bachofen, S. Schurch, Alveolar surface forces and lung architecture, Comparative Biochemistry and Physiology (2001), 183-193

P. Markart, C. Ruppert, M. Wygrecka, T. Colaris, B. Dahal, D. Wahnrath, H. Harbach, J. Wilhelm, W. Seeger, R. Schmidt, A. Guenther, Patients with ARDS show incomplete restoration of alveolar surface activity upon recombinant SP-C-based surfactant treatment: putative role of neutral lipids, Thorax (2007)

### SUMMARY OF THE INVENTION

The object of the invention consists of enhancing surfactants in a simple manner, in particular pulmonary surfactants, which, for example, are inhibited by cholesterol and exhibit dysfunction, where in particular the surface tension of the inhibited surfactants is improved by the treatment.

This object is solved by a method for enhancing a surfactant, in particular a pulmonary surfactant, which is further established in that the surfactant is enhanced with a lipid sequestrating or cholesterol sequestrating surfactant enhancement agent, wherein given, in particular neutral lipids or cholesterol of the surfactant are selectively sequestrated by means of the surfactant enhancement agent, such that the effect of the lipids and/or the effect of the cholesterol on the surfactant is reduced or reversed and thus also the dysfunction of the surfactant triggered by lipids or cholesterol.

### BRIEF DESCRIPTION OF THE DRAWINGS

FIG. 1 is graph that shows four slow compression/expansion cycles (A, B, C and D.

### DETAILED DESCRIPTION

In the context of the invention, the term “lipid sequestrating” or “cholesterol sequestrating” is understood to mean that lipids or cholesterol of a surfactant are removed from the surfactant in their particularly dysfunctional effect and/or are inhibited in their effect on the surfactant and/or act on the surfactant as a medium which makes lipids soluble in order to reduce or reverse the aforementioned inhibitory effect of the lipids and/or cholesterol. The surfactant dysfunction particularly occurs if there is an increased cholesterol level in the surfactant. The neutral lipids in the surfactant are also deemed responsible for dysfunction as a consequence of an increased level of lipids.

Through the action of the sequestrating surfactant enhancement agent, the dysfunction of the surfactant triggered by the cholesterol or by lipids, particularly neutral lipids, for example as a result of elevated cholesterol content or lipids above the corresponding normal level of cholesterol or lipids in the surfactants, is at least partially to entirely nullified as well as at least partially to completely reversed.

Lipid sequestrating or cholesterol sequestrating enhancement agents to be considered here are those media which particularly correspond to the action or active substance class of cyclodextrin, in particular of methyl-β-cyclodextrin, on a surfactant and/or a pulmonary surfactant. In this way, for example, the inhibiting effect of cholesterol on a surfactant is weakened or nullified.

Through the inventive enhancement of the surfactant, in particular endogenous surfactant, it is furthermore possible, for example, that a cholesterol-induced inhibiting effect of the cholesterol on the surfactant can be diagnosed. In addition to that, the properties of the surfactant can also be influenced or modified accordingly by the surfactant enhancement agent, which enables pulmonary diseases, in particular acute pulmonary diseases, to be treated. Examples of acute pulmonary illnesses are acute lung injury (ALI), ARDS, acute respiratory insufficiency, pneumonias, particularly respiration-induced pneumonias, nosocomial infections or systemic inflammatory response syndrome (SIRS) associated with ALI.

Through the use of the lipid sequestrating or cholesterol sequestrating surfactant enhancement agent, the surfactant is correspondingly influenced in its surface-active properties, so that the surface activity of endogenous, treated pulmonary surfactants can be determined, for instance, using a bubble surfactometer.

Moreover, the method is distinguished in that the surfactant is enhanced using a surfactant enhancement agent containing cyclodextrin or using a surfactant enhancement agent corresponding in action to a surfactant enhancement agent containing cyclodextrin or to the activity class of cyclodextrin.

Furthermore, an embodiment is envisaged in which an active substance of the surfactant enhancement agent methyl-β-cyclodextrin (MβCD) or cyclodextrin, especially 2-hydroxypropyl-β-cyclodextrin, or cyclodextrin derivatives which have 2-hydroxypropyl-β-cyclodextrin in particular, is present or dipalmitoylphosphatidylcholine (DPPC) or corresponding derivates of the aforementioned substances or a mixture of the substances mentioned or at least a lipid-selective and lipid-deactivating substance or at least a lipid-sequestrating substance are used.

In particular, the surface tension of the surfactant is reduced by the surfactant enhancement agent compared to an unenhanced surfactant or compared to the surfactant before enhancement.

In addition to that, it is envisaged in an embodiment that cholesterol of the surfactant and/or lipids, especially neutral lipids, of the surfactant are dissolved and/or passivated by the surfactant enhancement agent in a solution, particularly an aqueous solution. In this manner the inhibiting effect of lipids or cholesterol of the surfactant, for example on its surface activity, is treated accordingly. Consequently the surface activity and/or the surface tension of surfactants can be determined and diagnosed easily subsequent to enhancement.

Furthermore it is envisaged according to one embodiment that the surfactant enhancement agent sequestrate cholesterol and/or lipids of the surfactant which have an effect, particularly an inhibiting one, on the properties of the surfactant, especially the unenhanced surfactant, or which limit its properties.

In addition to that the object is solved by a method for producing a surfactant enhancement agent, in particular a pulmonary surfactant enhancement agent, which is further developed in that at least a portion of at least a lipid-selective and lipid-deactivating substance or at least a lipid-sequestrating or cholesterol-sequestrating substance will be or is mixed with the surfactant enhancement agent.

Preferably at least one substance will be or is mixed with the surfactant enhancement agent through which with the application on a surfactant, preferably an endogenous surfactant, cholesterol and/or preferably neutral lipids of the surfactant which have an effect, particularly an inhibiting one, on the properties of the surfactant, especially the unenhanced surfactant, or which limit its properties, are sequestrated.

Furthermore, in accordance with the invention, the surfactant enhancement agent is mixed with an active substance constituted by methyl-β-cyclodextrin (MβCD) or cyclodextrin, especially 2-hydroxypropyl-β-cyclodextrin, or cyclodextrin derivatives which have 2-hydroxypropyl-β-cyclodextrin in particular, or dipalmitoylphosphatidylcholine (DPPC) or corresponding derivates of the aforementioned substances or a mixture of the substances mentioned or at least a lipid-selective and lipid-deactivating substance or at least a lipid-sequestrating substance.

In addition to that, the surfactant enhancement agent can also contain other materials and/or active substances without loss of the aforementioned action of the sequestrating substance, i.e. the effect of this sequestrating substance is or will be retained. For example, during administration as a medication or pharmaceutical, another surfactant, for example an exogenously acting surfactant, can be added to the surfactant enhancement agent.

Furthermore, the object is solved by a surfactant enhancement agent, which is further developed in that at least a portion of at least a lipid-selective and lipid-deactivating substance or at least a lipid-sequestrating substance or cholesterol-sequestrating substance will be or is mixed with the surfactant enhancement agent.

Furthermore, the surfactant enhancement agent is distinguished in that with the use of the surfactant enhancement agent, i.e. during enhancement of a surfactant, the surfactant will be or is enhanced using the surfactant enhancement agent which has as an active substance methyl-β-cyclodextrin (MβCD) or cyclodextrin, especially 2-hydroxypropyl-β-cyclodextrin, or cyclodextrin derivatives which have 2-hydroxypropyl-β-cyclodextrin in particular, or dipalmitoylphosphatidylcholine (DPPC) or corresponding derivates of the aforementioned substances or a mixture of the substances mentioned or at least a lipid-selective and lipid-deactivating substance or at least a lipid-sequestrating substance.

In particular, the surface tension of the surfactant will be or is reduced with the use of the surfactant enhancement agent compared to an unenhanced surfactant or compared to the surfactant before enhancement.

Preferably cholesterol and/or lipids, especially neutral lipids, of the surfactant are dissolved and/or passivated by the surfactant enhancement agent in a solution, in particular an aqueous solution.

In addition to that it is envisaged that with the use of the surfactant enhancement agent, cholesterol and/or lipids, especially neutral lipids, of the particularly endogenous surfactant which have an effect, particularly an inhibiting one, on the properties of the surfactant, especially the unenhanced surfactant, or which limit its properties will be or are sequestrated by the surfactant enhancement agent.

Moreover, the invention is solved by the use of an enhancement agent for enhancing a surfactant, in particular an endogenous surfactant, in particular a pulmonary surfactant, in which the enhancement agent is or will be made according to the process described above, or in which the surfactant enhancement agent is made from the constituents described above. For this purpose, to avoid repetition, explicit reference is made to the information above.

Advantageously, the application is distinguished in that the enhancement agent is used as a therapeutic agent or pharmaceutical for treating pulmonary illnesses in living beings, in particular humans. For example, the enhancement agent can be present as a pharmaceutical in powder form for exhalative administration or in liquid form for intratrachial or intrabronchial administration. Beyond that in a further development it is possible that the enhancement agent be used with patients in aerosol form. Administration ensues in a manner known to the specialist, preferably by intratrachial instillation of a solution or suspension or in the form of an atomized solution or suspension or by atomization of powder.

Furthermore, the object is solved by the use of methyl-β-cyclodextrin (MβCD) or cyclodextrin, in particular 2-hydroxypropyl-β-cyclodextrin, or cyclodextrin derivatives which have 2-hydroxypropyl-β-cyclodextrin in particular, or dipalmitoylphosphatidylcholine (DPPC) or corresponding derivates of the aforementioned substances or a mixture of the substances mentioned or at least a lipid-selective and lipid-deactivating substance or at least a lipid-sequestrating substance, whereby cholesterol and/or lipids of a surfactant, in particular neutral lipids, are sequestrated, in particular for the production of pharmaceuticals for the treatment or early treatment of pulmonary illnesses in humans, in particular acute pulmonary illnesses.

Furthermore, the object is solved by the use of methyl-β-cyclodextrin (MβCD) or cyclodextrin, in particular 2-hydroxypropyl-β-cyclodextrin, or cyclodextrin derivatives which have 2-hydroxypropyl-β-cyclodextrin in particular, or dipalmitoylphosphatidylcholine (DPPC) or corresponding derivates of the aforementioned substances or a mixture of the substances mentioned or at least a lipid-selective and lipid-deactivating substance or at least a lipid-sequestrating substance, by which cholesterol and/or lipids of a surfactant, in particular neutral lipids, are sequestrated, in particular for the investigation, in particular diagnosis, of surfactants, in particular pulmonary surfactants.

In the context of the invention is furthermore possible that by the sequestrating effect of the surfactant enhancement agent the dysfunction will be reduced with respect to the surface tension of a surfactant, in particular an endogenous surfactant, whereby after corresponding enhancement of the surfactant the dysfunction is diminished or nullified by an increased level of cholesterol or lipids in the surfactant.

The invention is based on the idea that a use of methyl-β-cyclodextrin (MβCD) or other cyclodextrin or other cholesterol-sequestrating substances in the activity class or the active substance class of cyclodextrin, including dipalmitoylphosphatidylcholine (DPPC), and a use of substances corresponding to the activity class of cyclodextrin, which reverse or nullify the effect(s) of cholesterol by others means, including vitamin E, as agents makes it possible not only to diagnose or determine the effected or induced inhibition of the surfactant by cholesterol but also to treat the effected or induced inhibition of the surfactant by cholesterol in patients or people with the aforementioned symptoms or those with effected or induced inhibition of the surfactant by cholesterol or neutral lipids.

For the enhancement of the symptoms described, the substance or active substance can be delivered to the lung in aerosol form or instilled in the lung as a substance together with at least one exogenous, i.e. externally acting surfactant or delivered to the lung in another manner.

As a means of diagnosis, the inventive substance can be mixed with a specimen of a surfactant from a patient, the function of which is subsequently checked in a surface balance. Alternatively, the substance can be added to an aqueous solution or an aqueous phase of a surface balance to determine the function of the surfactants.

These results and findings were obtained on the basis of systems of model surfactants and pulmonary surfactants of CF patients and ARDS-patients. They will be explained below based on two examples.

### EXAMPLES

### Example 1

In a model study, 20 weight percent cholesterol was added to a cattle lipid surfactant extract in order to obtain an example of a surfactant charged with and/or inhibited by cholesterol; the function of the surfactant model was determined in a captive bubble surfactometer (CBS).

In the absence of methyl-β-cyclodextrin (MβCD), i.e. with unenhanced surfactant, the surface tension remained nearly unchanged near the equilibrium at 23 10⁻³N/m if the boundary surface or interface was reduced, whereby the surfactant is inhibited in a function.

In the presence of methyl-β-cyclodextrin (MβCD) (20 mmol), i.e. with enhanced surfactant, in the aqueous phase the surfactant regained its normal function and the surface tension receded to almost zero.

### Example 2

In the study with specimens from CF patients, surfactants obtained by means of bronchoalveolar lavage (BAL) were also examined with the captive bubble surfactometer (CBS).

In the absence of methyl-β-cyclodextrin (MβCD), the surface tension remained near the equilibrium (23 10⁻³N/m) if the boundary surface or interface was reduced.

In the presence of methyl-β-cyclodextrin (MβCD) (20 mMol) in the aqueous phase, the surfactant recovered its function almost entirely or completely. After three hours in the presence of methyl-β-cyclodextrin, the films reduce their surface tensions almost to zero to an area reduction in a normal manner.

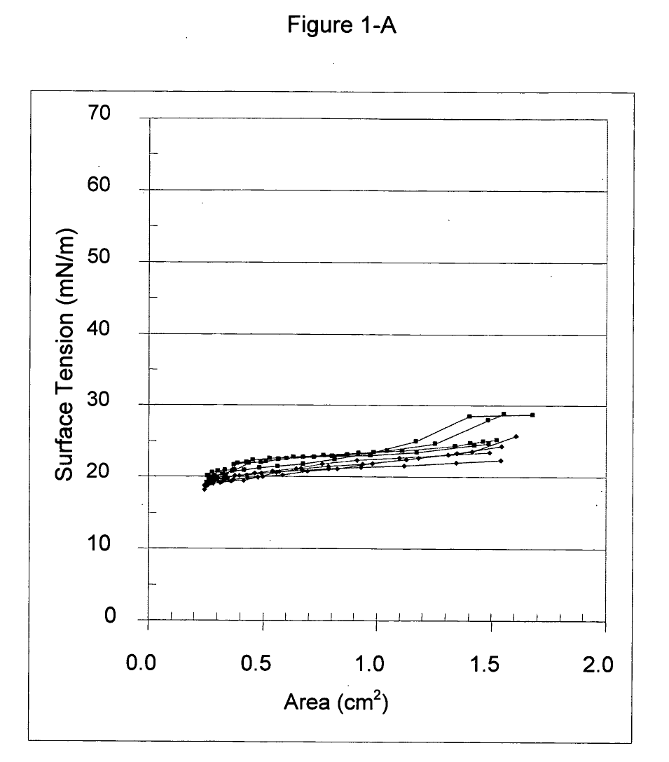

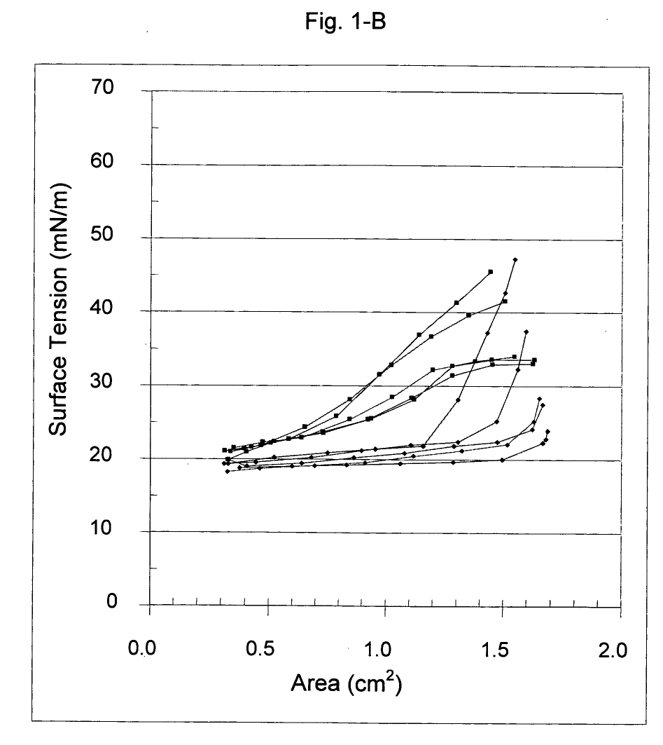

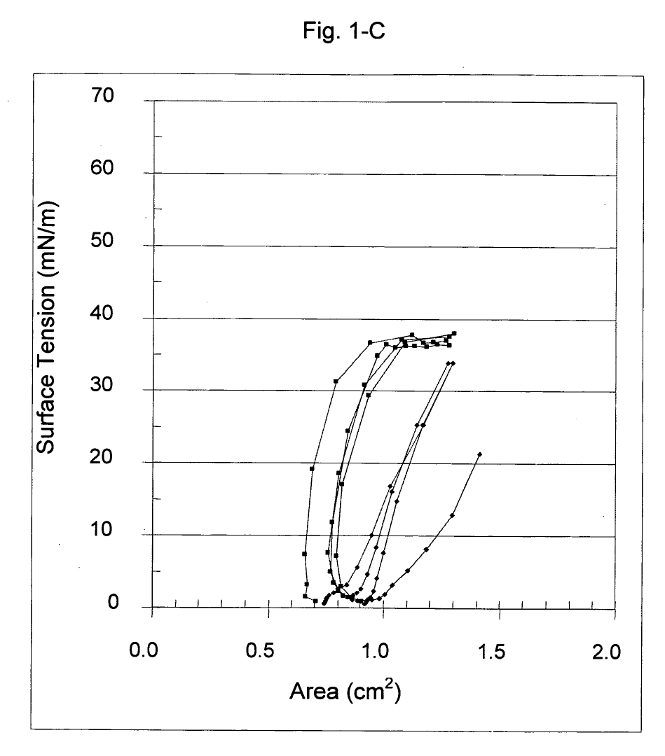

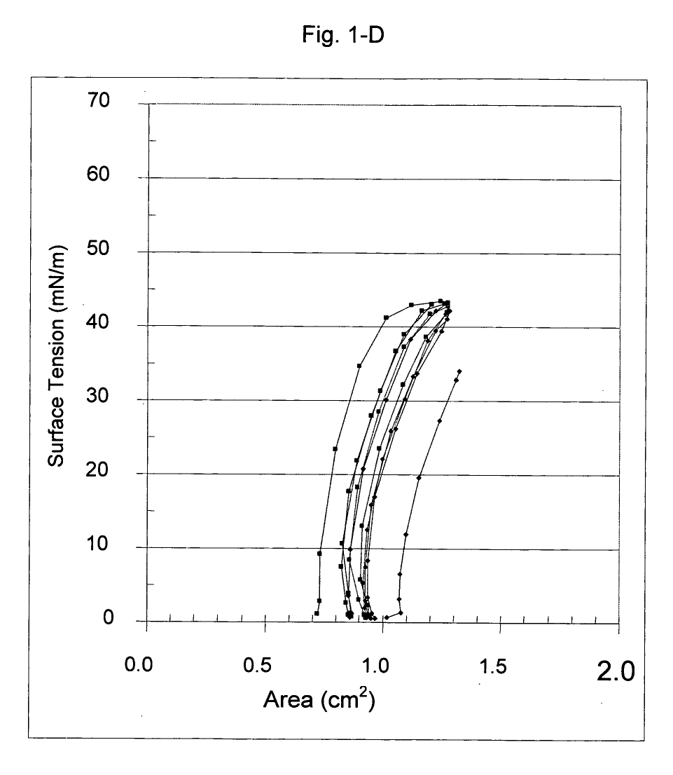

Furthermore, in FIG. 1 measurements of surface tensions are shown for ranges of surfactants which were obtained from a bronchoalveolar fluid of a CF patient (cystic fibrosis patient) in a captive bubble surfactometer (CBS).

The left side of FIG. 1 shows four slow compression/expansion cycles (A and C). On the right side, cycles (B and D) are shown in which the ranges were reduced and enlarged for a normal rate of respiration.

The upper cycles (A and B) show a strongly inhibited surfactant in which the surface tension remains about 20 mN/m. Under such conditions, the alveolar structure of the lung is not maintained and the area in which gas exchange takes place is reduced by half.

The lower cycles (C and D) illustrate the effect of methyl-β-cyclodextrin (MβCD) (20 mmol) on the (inhibited) surfactant in the aqueous phase. The surface tension of these inventively enhanced surfactants drops to nearly zero. These isotherms (C and D) do not differ from fully functional (non-inhibited) surfactants.

In summary it is apparent that the results with surfactants obtained from bronchoalveolar liquids of a CF patient make it clear that surfactants of CF patients can be inhibited and that the inhibition can be nullified or reversed by methyl-β-cyclodextrin (MβCD). Here it is to be assumed that the surfactants of CF patients are inhibited by cholesterol.

1-18. (canceled)

19: A method for enhancing a surfactant, comprising
adding to the surfactant a surfactant enhancement agent that is configured to sequestrate lipids or cholesterol, such that the performance of the surfactant when administered to a patient is enhanced.

20: The method of claim 19, wherein the surfactant enhancement agent is cyclodextrin or derivatives thereof.

21: The method according to claim 19, wherein the surfactant enhancement agent is methyl-β-cyclodextrin (MβCD) or derivatives thereof.

22: The method of claim 19 wherein the surfactant enhancement agent is a 2-hydroxypropyl-β-cyclodextrin or derivatives thereof.

23: The method of claim 19, wherein the surface tension of the surfactant is reduced when combined with the surfactant enhancement agent compared to the surfactant before enhancement.

24: The method of claim 19, wherein the surfactant enhancement agent dissolves lipids or cholesterol in an aqueous solution.

25: A method for producing a surfactant enhancement agent, in particular a pulmonary surfactant enhancement agent, comprising the step of mixing at least a portion of a lipid-sequestrating or cholesterol-sequestrating substance with the surfactant enhancement agent.

26: The method according to claim 25, wherein the characterized in that at least one substance will be or is mixed with the surfactant enhancement agent through which cholesterol and/or preferably neutral lipids of the surfactant which have an effect, particularly an inhibiting one, on the properties of the surfactant, especially the unenhanced surfactant, or which limit its properties, are sequestrated.

27: The method according to claim 25, characterized in that methyl-β-cyclodextrin (MβCD) or derivatives thereof are mixed with the surfactant enhancement agent.

28: A composition comprising a lipid-selective and lipid-deactivating substance and the surfactant enhancement agent.

29: The composition of claim 28, wherein the surfactant enhancement agent is cyclodextrin.

30: The composition of claim 28, wherein the surface tension of the surfactant will be or is reduced with the use of the surfactant enhancement agent compared to an unenhanced surfactant.

31: The composition of claim 28, wherein cholesterol and/or lipids of the surfactant are or will be passivated by the surfactant enhancement agent in an aqueous solution.

32: The composition of claim 28, wherein characterized in that with the use of the surfactant enhancement agent, cholesterol and/or lipids of the surfactant which have an effect, particularly an inhibiting one, on the properties of the surfactant, especially the unenhanced surfactant, or which limit its properties will be or are sequestrated by the surfactant enhancement agent.

33: A method of treating a patient with a respiratory distress syndrome, the method comprising the step of administering to a patient a composition comprising a therapeutically effective amount of a pulmonary surfactant and an enhancement agent made according to the process of claim 25 in an amount sufficient to reduce the surface tension of the surfactant compared to an unenhanced surfactant.

34: The method of claim 33, wherein the enhancement agent is used as a therapeutic agent or pharmaceutical for treating infant respiratory distress syndrome.

17: The method of claim 33, wherein the enhancement agent is used as a therapeutic agent or pharmaceutical for treating acute respiratory distress syndrome.
